(19) United States
(12) Patent Application Publication
(10) Pub. No.: US 2025/0144207 A1
(43) Pub. Date: May 8, 2025

(54) COMBINATION THERAPY COMPRISING ANTI-CCR9 ANTIBODY AND VINCRISTINE FOR CANCER

(71) Applicants: SUNROCK BIOPHARMA S.L., Santiago de Compostela (ES); CONSEJO SUPERIOR DE INVESTIGACIONES CIENTÍFICAS, Madrid (ES)

(72) Inventors: Silvia Santamaría García-Minguillán, Madrid (ES); Marisa Delgado Álvarez, Madrid (ES); José Alberto García Sanz, Madrid (ES); Leonor Judith Kremer Barón, Madrid (ES); Laureano Simón Buela, Santiago de Compostela (ES); Pablo Garrido Cuesta, Santiago de Compostela (ES); Amparo Pérez Díaz, Santiago de Compostela (ES)

(73) Assignees: SUNROCK BIOPHARMA S.L., Santiago de Compostela (ES); CONSEJO SUPERIOR DE INVESTIGACIONES CIENTÍFICAS, Madrid (ES)

(21) Appl. No.: 18/835,297

(22) PCT Filed: Feb. 3, 2023
(86) PCT No.: PCT/EP2023/052720

§ 371 Date: Aug. 1, 2024

(30) Foreign Application Priority Data
Feb. 3, 2022 (EP) 22382093.7

(51) Int. Cl. A61K 39/395; A61K 31/475; A61K 31/573; A61K 39/00; A61P 35/00

(52) U.S. Cl. CPC A61K 39/3955; A61K 31/475; A61K 31/573; A61P 35/00; A61K 2039/505

(57) ABSTRACT

The invention provides an anti-CCR9 antibody molecule for use in a method of treatment of cancer in a mammalian subject wherein the anti-CCR9 antibody molecule is administered simultaneously, sequentially or separately with a chemotherapeutic agent selected from the group consisting of: vincristine, docetaxel, paclitaxel, nanoparticle albumin-bound paclitaxel, and vinblastine, wherein said anti-CCR9 antibody molecule and said chemotherapeutic agent are not conjugated together. Also provided are related methods of treatment.

This application claims priority from EP22382093.7 filed 3 Feb. 2022, the contents and elements of which are herein incorporated by reference for all purposes.

### Field of the Invention

The present invention relates to therapeutic molecules and their use in combination to treat cancer.

### Background to the Invention

Chemokines are a family of small structurally related proteins that bind to seven transmembrane G protein-coupled receptors, with predominantly chemotactic function, currently composed of 44 members in humans. Chemokines and their receptors have an essential role in organogenesis and lymphocyte trafficking, in both homeostatic and inflammatory conditions. Chemokines generate gradients, either soluble or immobilized, and are responsible for tasks as diverse as directing cell movement, stimulating cell growth, activation or differentiation, or even regulating organogenesis in mammals (Zlotnik and Yoshie 2000, Raman, Sobolik-Delmaire et al. 2011). Regarding the cells of the immune system, chemokines play a key role in homeostasis and in both innate and acquired immunity, by controlling the trafficking and recruitment of leukocytes.

Chemokines exert their biological effects through their interaction with specific receptors present on the cell surface. Structurally, these receptors belong to the G-protein-coupled seven-transmembrane-domain receptor (GPCR) superfamily (Bachelerie, Ben-Baruch et al. 2014). This system is characterized by having certain redundancy, so the same chemokine can bind to multiple receptors, and vice versa, and they are key molecules in the activation of different genes, giving rise to different cellular responses that include chemotaxis, cell survival and proliferation (DeVries, Kelvin et al. 2006). There is a strong association between aberrant tumor cell expression of chemokine receptors such as CXCR4 or CCR7 and cancer progression, organ-selective metastasis, and poor prognosis.

At the same time, they have great potential for use in tumor-targeted therapy (Weitzenfeld and Ben-Baruch 2014) in which, cancer-related chemokines could promote tumor proliferation, angiogenesis, and chemoresistance (Lazennec and Richmond 2010, Mukaida and Baba 2012, Sarvaiya, Guo et al. 2013).

Human chemokine receptor CCR9 (GenBank accession number. U45982) is a member of this superfamily of receptors, identified by Zaballos et al. (Zaballos, Gutiérrez et al. 1999) (EMBL database accession number AJ132337) and Youn et al. (Youn, Kim et al. 1999). Under physiological conditions, the expression of CCR9 is highly restricted: it has been described in thymocytes (Zaballos, Gutiérrez et al. 1999, Carramolino, Zaballos et al. 2001), infiltrating immune cells in the small intestine (Kunkel, Campbell et al. 2000) and in small subpopulations of circulating memory T cells (Zabel, Agace et al. 1999) and plasmacytoid dendritic cells (Wendland, Czeloth et al. 2007).

Unlike other chemokine receptors, CCR9 has a single ligand called CCL25 (Chemokine Ligand 25 or TECK) secreted by epithelial and dendritic cells from the thymus and the small intestinal crypt epithelium and that, when binding to its receptor, it activates intracellular signaling pathways related to cell survival and mobility (Wurbel, Malissen et al. 2006). The CCR9-CCL25 interaction is a key regulator of thymocyte migration in thymus and of cell homing to the intestinal tract. Recent insights into the mechanisms of CCL25/CCR9 show that they are involved in tumor chemoresistance and metastasis (Tu, Xiao et al. 2016), and the potential application of CCR9 in targeted therapies.

In cancer, CCR9 expression has been increased in CD4+ helper T cells in acute and chronic lymphocytic leukemias (Qiuping, Qun et al. 2003) and in other types of hematological tumors such as follicular lymphoma and diffuse B-cell lymphoma (Wu, Doan et al. 2014). At the same time, aberrant expression of CCR9 has been described in some solid tumors such as prostate, breast, pancreatic and melanoma cancer (Letsch, Keilholz et al. 2004, Amersi, Terando et al. 2008, Johnson, Singh et al. 2010, Singh, Stockard et al. 2011, Heinrich, Arrington et al. 2013, Gupta, Sharma et al. 2014). This expression of CCR9 would be advantageous for cells since, by binding to its ligand, CCL25, different signaling pathways would be activated, including PI3K/Akt, increasing the cell survival of transformed T lymphocytes and resistance to apoptosis mediated by this signaling pathway in different types of cancer (Sharma, Singh et al. 2010, Johnson-Holiday, Singh et al. 2011), activates the JNK1 anti-apoptotic pathway, and enhances proliferation by activating Notch1 in leukemia cells, especially in T-lineage acute lymphoblastic leukemia (T-ALL) (Mirandola, Chiriva-Internati et al. 2012). In addition, moderate levels of CCR9 expression have been observed in T cell chronic lymphocytic leukemia (T-CLL) CD4+ cells. Notably, when CCR9 is internalized into T-ALL CD4+ T cells, the chemotactic and adhesive ability of the leukemia cells are eliminated, indicating that CCR9 is closely associated with the infiltration and metastasis of leukemia cells.

The PI3K/AKT pathway is one of the most important signaling pathways related to multiple physiological functions of cells and the development of certain diseases (Thorpe, Yuzugullu et al. 2015). Sharma et al. found that the interaction between CCR9 and its natural ligand CCL25 upregulated levels of PI3K, AKT, ERK1/2 and GSK-3B to exert antiapoptotic effects while downregulating proapoptotic proteins, such as caspase-3 levels in pancreatic cancer cells, thereby inhibiting cell apoptosis. However, PI3K inhibitors (wortmannin) can significantly downregulate these CCR9-mediated antiapoptotic proteins in pancreatic cancer cells, suggesting that the antiapoptotic effect of CCR9 is primarily regulated by PI3K. In addition, CCL25 is capable of inhibiting the cytotoxic effects of etoposide (an antitumor drug) in tumor-bearing mice. However, when the CCR9 monoclonal antibody is used to block the CCR9/CCL25 interaction, this cytotoxic inhibition is abolished (Sharma, Singh et al. 2010). Moreover, the combination of a CCL25 neutralizing antibody and etoposide in the treatment of tumor burden results in significant tumor reduction compared with that of single-drug treatment. These results indicate that the mechanism by which the CCR9/CCL25 axis regulates PI3K/AKT-dependent antiapoptotic signaling in pancreatic cancer cells may contribute to the low number of apoptotic cells and the mild chemotherapy response. Matrix metalloproteinases (MMPs) constitute a class of zinc-dependent endogenous proteases that play important roles in the remodeling of tissue and the degradation of various proteins in the extracellular matrix (Mittal, Patel et al. 2016), promoting cell proliferation, migration and differentiation and playing a role in apoptosis, angiogenesis, tissue repair and immune response (Raffetto and Khalil 2008). In addition, MMPs are also involved in a key step in the processes of cancer cell invasion and metastasis (Yoon, Park et al. 2003).

These results suggest that the expression and activation of CCR9/CCL25 promote cancer cell migration, invasion, and MMP expression, which together may affect pancreatic cancer metastasis (Singh, Singh et al. 2004). Taken together, these findings suggest that knocking out or blocking CCR9/CCL25 can beneficially affect the clinical treatment of pancreatic cancer.

CCR9 is highly expressed in the breast cancer MDA-MB-231 cell line. Although the activation of CCR9/CCL25 signaling did not significantly increase the migration of MDA-MB-231 cells, it could significantly promote the invasion of MDA-MB-231 cells (Zhang, Sun et al. 2016). Activation of CCR9/CCL25 signaling increased MDAMB-231 cell invasion by significantly upregulating MMP-1 expression and moderately upregulating MMP-2 and MMP-11 expression. Notably, the downregulation of E-cadherin and the upregulation of N-cadherin and Vimentin have been implicated in the increased motility and migration of cancer cells (Cavallaro and Christofori 2004, Berx and van Roy 2009). Nevertheless, CCR9/CCL25 signaling slightly decreased not only the expression of E-cadherin but also the expression of N-cadherin and Vimentin, which might partly explain why CCR9/CCL25 signaling had no significant effect on the migration of MDA-MB-231 cells. These results indicate that CCR9/CCL25 signaling promotes the invasion of breast cancer cells by regulating multiple epithelial-mesenchymal transition (EMT) markers, and CCR9/CCL25 signaling may be a potential target for blocking breast cancer cell invasion.

CCR9/CCL25 interactions are showed to promote breast cancer cell (MDA-MB-231) proliferation and upregulate antiapoptotic signaling, which is mediated by the PI3K/AKT survival pathway and is independent of FAK (Johnson-Holiday, Singh et al. 2011). In addition, another study also found that the expression of CCR9 was significantly increased in moderately and poorly differentiated breast cancer tissues compared with its level of expression in nonneoplastic breast tissue. Interestingly, CCR9 expression was significantly higher in poorly differentiated breast cancer tissues than it was in moderately differentiated breast cancer tissues. Similarly, CCR9 was highly expressed in the aggressive breast cancer MDA-MD-231 cell line compared to its expression level in the less aggressive breast cancer MCF-7 cell line. In summary, CCR9 is functionally and significantly expressed in breast cancer tissue and cells, and CCL25 activation promotes breast cancer cell migration and invasion and MMP expression, which are key components of breast cancer metastasis.

Specific therapeutic tools to treat human CCR9+ tumors growing in xenogenic models are limited to the use of toxin-coupled ligands (CCL25-PE38 fusion protein) (Hu, Zhang et al. 2011) or ligand-specific antibodies alone or combined with the cytotoxic agent etoposide (Sharma, Singh et al. 2010). In these strategies, the CCL25-CCR9 interaction is targeted to eliminate tumor cells; although the results have been limited, they provide evidence that CCR9 is a potential target for cancer immunotherapy.

Given the lack of therapies targeted to CCR9, there is still a need in the art to provide agents recognizing CCR9 specifically that are suitable for the diagnosis, prognosis and/or treatment of a disease or condition concomitant with cells expressing CCR9. Recently, it was found that neither human nor mouse triple negative breast cancers (TNBCs) express CCL25 (Chen, Cong et al. 2020). In addition, another study found that the success of T cell-based cancer immunotherapy is limited by tumor's resistance against killing by cytotoxic T lymphocytes (Brightman, Naradikian et al. 2020). Tumor-immune resistance is mediated by cell surface ligands that engage immune-inhibitory receptors on T cells (Borst, Ahrends et al. 2018). These ligands represent potent targets for therapeutic inhibition. CCR9 is expressed in many cancers and exerts strong immune-regulatory effects on T cell responses in multiple tumors (Khandelwal, Breinig et al. 2015). Unlike PD-L1, which inhibits T cell receptor signaling, CCR9 regulates STAT signaling in T cells, resulting in reduced T-helper-1 cytokine secretion and reduced cytotoxic capacity. Moreover, inhibition of CCR9 expression on tumor cells facilitated the immunotherapy of human tumors by tumor-specific T cells in vivo. In summary, these studies have elucidated the immunobiological roles of CCR9/CCL25 axis in cancer immunity and immunotherapy, providing a certain experimental basis for further research on cancer immunotherapy.

WO2015/075269A1 discloses anti-CCR9 antibodies and their use in the treatment of cancers, including T cell acute lymphoblastic leukaemia (T-ALL), prostate cancer, breast cancer, melanoma, ovarian cancer, colorectal cancer and lung cancer. WO2015/075269A1 also discloses combination therapy with anti-CCR9 antibodies and antagonists of CCR9 such as those described in US2005/0049286 and the CCL25-PE38 fusion protein. The anti-CCR9 antibodies and the other drugs may form part of the same composition, or be provided as a separate composition for administration at the same time or at a different time. WO2015/075269A1 also discloses immunoconjugates in which the anti-CCR9 antibody is conjugated to or in the form of a fusion protein with another therapeutic agent such as a chemotherapeutic agent, including vincristine.

There remains an urgent unmet medical need for effective therapies with which to treat cancer. Despite the advances described above, there remains a need for therapeutic strategies for the treatment cancer, including T-cell acute lymphoblastic leukaemia (T-ALL) and pancreatic cancer, which increase survival time still further. The present invention seeks to address this need and, as described in detail herein, provides further related advantages.

### BRIEF DESCRIPTION OF THE INVENTION

The present invention relates generally to therapeutic agents and their use for treatment of cancer in mammalian subjects, particularly cancers such as T-ALL.

As described herein, the present inventors surprisingly found that combination treatment of tumor-bearing mice with a CCR9-targeted antibody (SRB1) in combination with the chemotherapy agent vincristine resulted in significantly increased median survival time not only in comparison with controls, but also in comparison with treatment groups that received only the SRB1 antibody or only vincristine. The results described herein show a synergistic relationship between SRB1 and vincristine in terms of anti-tumor efficacy. Moreover, the significant increase in median survival time was also achieved with a triple combination treatment using the SRB1 antibody, vincristine and the corticosteroid dexamethasone. Without wishing to be bound by any particular theory, the present inventors believe that the synergistic relationship on anti-tumor efficacy seen with the SRB1 antibody and vincristine will also be exhibited by other anti-CCR9 antibodies in combination with other vinca alkaloids as described herein.

The dosing schedule and optimal form of administration of the proposed combination will be determined by the different chemical composition and the inherent different half-lives of the compounds: an anti-CCR9 antibody molecule and a chemotherapeutic agent, as defined herein. Therefore, following clinical guidelines, separate and/or sequential administration is recommended (depending on the case). Formation of antibody-drug conjugates may be considered less preferred, because the optimum frequency of administration of both agents may be different, as antibodies have longer half-life, they can be administered weekly or less frequently and the chemotherapeutic agent may require more frequent dosing to maintain high concentration.

Accordingly, in a first aspect the present invention provides an anti-CCR9 antibody molecule for use in a method of treatment of a cancer in a mammalian subject, wherein the anti-CCR9 antibody molecule is administered simultaneously, sequentially or separately with a chemotherapeutic agent, such as a chemotherapeutic agent selected from: a vinka alkaloid (e.g. vincristine), docetaxel, paclitaxel, nanoparticle albumin-bound paclitaxel, and vinblastine, wherein said anti-CCR9 antibody and said chemotherapeutic agent are not conjugated together. In certain embodiments, the anti-CCR9 antibody molecule may be for use in method that further comprises simultaneously, sequentially or separately administering a corticosteroid, such as dexamethasone.

In some embodiments the anti-CCR9 antibody molecule comprises an anti-CCR9 monoclonal antibody or antigen-binding fragment thereof that specifically binds to CCR9. In particular, the anti-CCR9 monoclonal antibody or antigen-binding fragment thereof may be selected from the group consisting of: Fv, Fab, F(ab′)₂, Fab′, ScFv, scFv-Fc, minibody, nanobody and diabody.

In some embodiments the antibody or antigen-binding fragment thereof may comprise:
a heavy chain complementarity determining region 1 (CDR-H1) comprising the amino acid sequence: NEWMN (SEQ ID NO: 1) or KFWMN (SEQ ID NO: 2);
a heavy chain complementarity determining region 2 (CDR-H2) comprising the amino acid sequence: EIRLKSNNYATHYAESVKG (SEQ ID NO: 3);
a heavy chain complementarity determining region 3 (CDR-H3) comprising the amino acid sequence: DGWFAY (SEQ ID NO: 4);
a light chain complementarity determining region 1 (CDR-L1) comprising the amino acid sequence: RSSQSLLHSNGNTYVQ (SEQ ID NO: 5) or RSSQSLVHSNGNTYLN (SEQ ID NO: 6);
a light chain complementarity determining region 2 (CDR-L2) comprising the amino acid sequence: KVSNRFP (SEQ ID NO: 7) or KVSNRFS (SEQ ID NO: 8); and
a light chain complementarity determining region 3 (CDR-L3) comprising the amino acid sequence: AQSTHVPRT (SEQ ID NO: 9) or SQSTHEPRT (SEQ ID NO: 10).

In particular embodiments, the six CDR sequences may be selected according to the “91R” antibody disclosed in FIG. 9 of WO2015/075269 (incorporated herein by reference). In this case, the CDR sequences are as follows: CDR-H1 is SEQ ID NO: 1, CDR-H2 is SEQ ID NO: 3, CDR-H3 is SEQ ID NO: 4, CDR-L1 is SEQ ID NO: 5, CDR-L2 is SEQ ID NO: 7 and CDR-L3 is SEQ ID NO: 9.

In particular embodiments, the six CDR sequences may be selected according to the “92R” antibody disclosed in FIG. 9 of WO2015/075269 (incorporated herein by reference). In this case, the CDR sequences are as follows: CDR-H1 is SEQ ID NO: 2, CDR-H2 is SEQ ID NO: 3, CDR-H3 is SEQ ID NO: 4, CDR-L1 is SEQ ID NO: 6, CDR-L2 is SEQ ID NO: 8 and CDR-L3 is SEQ ID NO: 10.

In some embodiments the antibody or antigen-binding fragment thereof comprises:

a heavy chain variable region comprising the
amino acid sequence:
(SEQ ID NO: 11)
EVKLEDSGGGLVQPGRSMKLSCVASGFTFSNFWMNWVRQSPEKGLEWVA
EIRLKSNNYATHYAESVKGRFTISRDDSKSSVYLQMNNLRTEDTGIYYC
TSDGWFAYWGQGTLVTVSA
or
(SEQ ID NO: 12)
EVKLEESGGGLVQPGGSMKLSCVASGFTFNKFWMNWVRQSPEKGLEWVA
EIRLKSNNYATHYAESVKGRFTISRDDSKSSVYLQMNNLRAEDTGIYYC
ASDGWFAYWGQGTLVTVSA
or
(SEQ ID NO: 13)
EVQLVESGGGLVKPGGSLRLSCAASGFTFSKFWMNWVRQAPGKGLEWVG
EIRLKSNNYATHYAESVKGRFTISRDDSKNTLYLQMNSLKTEDTAVYYC
TSDGWFAYWGQGTLVTVSS;
and
a light chain variable region comprising the
amino acid sequence:
(SEQ ID NO: 14)
DVVMTQTPLSLPVSLGDQTSISCRSSQSLLHSNGNTYVQWYLRKPGQSP
KLLIYKVSNRFPGVPDRFSGSGSGTDFTFKISRVEAEDLGVYFCAQSTH
VPRTFGGGTKLEIKR
or
(SEQ ID NO: 15)
DVVMTQTPLSLPVSLGDQASISCRSSQSLVHSNGNTYLNWCLQRPGQSP
KSLIYKVSNRFSGVPDRFSGSGSGTDFTLKISRVEAEDLGVYFCSQSTH
FPRTFGGGTKLEIKR
or
(SEQ ID NO: 16)
DVQMTQSPSSLSASVGDRVTITCRSSQSLVHSNGNTYLNWYQQKPGKAP
KLLIYKVSNRFSGVPSRFSGSGSGTDFTLTISSLQPEDFATYYCSQSTH
FPRTFGGGTKVEIK.

In particular, the antibody or antigen-binding fragment thereof may comprise the VH and VL of SEQ ID NOs: 11 and 14, or of SEQ ID NOs: 12 and 15, or of SEQ ID NOs: 13 and 16.

In some embodiments the antibody or antigen-binding fragment thereof exhibits a binding affinity dissociation constant K_{D} of 500 nM, 250 nM, 100 nM, 10 nM, or lower for human CCR9. The K_{D} value may be determined by any appropriate method. In particular, the K_{D} value may be as determined by Surface Plasmon Resonance (SPR) (e.g. BIACORE). In certain cases, the binding affinity constant K_{D} may be as determined by, or inferred from, whole cell-based antibody binding determination.

In some embodiments the antibody or antigen-binding fragment thereof exhibits CCR9 specific binding in the presence of 10 μg/mL concentration of human CCL25, as measured by flow cytometry. The present inventors have found that certain anti-CCR9 antibodies, such as “91R” and “92R” as disclosed in WO2015/075269 exhibit CCR9 binding that is essentially unaffected by the presence of the ligand CCL25. This contrasts with an anti-CCR9 antibody such as “3C3” disclosed in WO00/53635 which displays significant sensitivity to the presence of CCL25. Indeed, at physiologically relevant concentrations of CCL25 the binding of the antibody “3C3” to CCR9 is effectively abolished. The “SRB1” antibody having the VH and VL of SEQ ID NOS: 13 and 16, respectively has also been found to exhibit CCR9 binding in the presence of CCL25. Therefore, like “91R” and “92R”, the “SRB1” is preferred for use in the treatment of a condition in which CCL25 concentration may be elevated, including in the treatment of certain cancers as described in detail herein.

In some embodiments the antibody or antigen-binding fragment thereof exhibits CCR9+ lymphocyte depletion. The present inventors have, for example, observed antibody mediated complement-dependent cytotoxicity (CDC) utilizing anti-CCR9 antibodies such as “91R”, “92R” and “SRB1” described above. In certain embodiments, the anti-CCR9 antibody or antigen-binding fragment thereof may be an antibody isotype or sub-isotype that exhibits or has been engineered to exhibit CDC effector function (for example, for IgG in humans: hIgG3>hIgG1>hIgG2>hIgG4; in mice: mIgG2a>mIgG1); also IgM displays good CDC).

In some embodiments the chemotherapeutic agent comprises docetaxel, paclitaxel, nanoparticle albumin-bound paclitaxel, vinblastine and/or vincristine.

In some embodiments the chemotherapeutic agent comprises a vinka alkaloid, such as vincristine, vinblastine, vindesine, vinorelbine, vincaminol, vineridine, and/or vinburnine.

In some embodiments the simultaneous, sequential or separate administration further comprises administration of a corticosteroid such as dexamethasone.

In some embodiments both a vinka alkaloid (e.g. vincristine) and a corticosteroid (e.g. dexamethasone) are administered with the anti-CCR9 antibody molecule.

In some embodiments the subject is a mammal (preferably a human) who has been diagnosed as having, or as being at risk of developing, a cancer. In some embodiments, the subject has been or is being treated with anti-cancer therapy (e.g. with an anti-cancer drug treatment, surgical treatment and/or radiotherapy). In particular cases, the cancer of the subject may have relapsed, spread and/or developed resistance to a first-line therapy.

In some embodiments the cancer of the subject is a blood neoplasia.

In some embodiments the cancer of the subject is selected from the group: T-cell acute lymphoblastic leukemia (T-ALL), T-cell lineage lymphomas, and acute myeloid leukemia (AML).

In a second aspect the present invention provides a chemotherapeutic agent for use in a method of treatment of a cancer in a mammalian subject, wherein the chemotherapeutic agent is administered simultaneously, sequentially or separately with an anti-CCR9 antibody molecule, and wherein said chemotherapeutic agent is selected from the group consisting of: a vinka alkaloid (e.g. vincristine), docetaxel, paclitaxel, nanoparticle albumin-bound paclitaxel, and vinblastine, and wherein said chemotherapeutic agent and said anti-CCR9 antibody molecule are not conjugated together. In some embodiments, the method may further comprise simultaneously, sequentially or separately administering a corticosteroid, such as dexamethasone.

The anti-CCR9 antibody molecule may be as defined in accordance with the first aspect of the invention.

The chemotherapeutic agent may be as defined in accordance with the first aspect of the invention.

The subject and/or the cancer may be as defined in accordance with the first aspect of the invention.

In a third aspect the present invention provides a method of treatment of cancer in a mammalian subject, comprising administering simultaneously, sequentially or separately to the subject in need thereof a therapeutically effective amount of an anti-CCR9 antibody molecule and a chemotherapeutic agent selected from the group consisting of: a vinka alkaloid (e.g. vincristine), docetaxel, paclitaxel, nanoparticle albumin-bound paclitaxel, and vinblastine, wherein said anti-CCR9 antibody molecule and said chemotherapeutic agent are not conjugated together. In some embodiments, the method may further comprise simultaneously, sequentially or separately administering a corticosteroid, such as dexamethasone.

The anti-CCR9 antibody molecule may be as defined in accordance with the first aspect of the invention.

The chemotherapeutic agent may be as defined in accordance with the first aspect of the invention.

The subject and/or the cancer may be as defined in accordance with the first aspect of the invention.

In a fourth aspect the present invention provides use of an anti-CCR9 antibody molecule in the preparation of a medicament for use in a method of treatment of cancer in a mammalian subject, wherein said method is as defined in accordance with the third aspect of the invention.

In a fifth aspect the present invention provides use of a chemotherapeutic agent in the preparation of a medicament for use in a method of treatment of cancer in a mammalian subject, wherein said method is as defined in accordance with the third aspect of the invention, and wherein said chemotherapeutic agent is as defined in accordance with the first aspect of the invention.

The invention includes the combination of the aspects and preferred features described except where such a combination is clearly impermissible or expressly avoided.

These and further aspects and embodiments of the invention are described in further detail below and with reference to the accompanying examples and figures.

### BRIEF DESCRIPTION OF THE FIGURES

FIG. 1 shows optimization of SRB1 antibody doses (16 mg/kg) and its effect on the survival of NSG mice with MOLT4-GFP xenografts. (A) Experimental design. Briefly NSG mice were grafted with MOLT-4 cells (1×10⁶ cells/mice; I.V) at day 0, at day 4 16 mg/kg of SRB1 or Isotype were administered I.P. once weekly for four weeks, mouse status was checked daily. (B) Cummulative survival graph of the mice.

FIG. 2 shows in vivo efficacy of anti-CCR9 antibody SRB1 (4 mg/kg) in NSG mice with MOLT4-GFP xenografts—Combination of suboptimal doses of SRB1 with chemotherapy agents. (A) NSG mice were grafted with MOLT-4 cells (1×10⁶ cells/mice; I.V) at day 0 and at days 4, 10, 17 and 24, 4 mg/kg of SRB1, PBS or Isotype were administered I.P. (B) Besides groups described in (A), 0.6 mg/kg of vincristine were administered on day 5, or 50 μg/mouse/dose of dexamethasone in days 5-9 (C) or both (D).

FIG. 3 shows the effect of anti-CCR9 antibody SRB1 (4 mg/kg) in combination with chemotherapy agents on survival of NSG mice with MOLT4-GFP xenografts.

### DETAILED DESCRIPTION OF THE INVENTION

Aspects and embodiments of the present invention will now be discussed. Further aspects and embodiments will be apparent to those skilled in the art. All documents mentioned in this text are incorporated herein by reference.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### CCR9

Chemokines are a family of small, structurally related proteins that bind to seven transmembrane spanning G protein-coupled receptors. Chemokines and their receptors have an essential role in organogenesis and lymphocyte trafficking, in both homeostatic and inflammatory conditions.

The amino acid sequence of CCR9 (C-C chemokine receptor type 9) is disclosed at the UniProt accession number P51686 (see version 2 of 5 Sep. 2006), the entire contents of which is incorporated herein by reference.

### Anti-CCR9 Antibody Molecule

The anti-CCR9 antibody molecule binds to, preferably specifically binds to CCR9 (e.g. human CCR9). In certain embodiments the anti-CCR9 antibody molecule comprises a monoclonal antibody or antigen-binding fragment thereof that specifically binds to CCR9.

“Specifically binds” in this context may be distinguished from non-specific binding by virtue of the degree of binding affinity and/or the selectivity of binding, wherein the binding affinity for CCR9 is greater than for other antigens. The anti-CCR9 antibody or antigen-binding fragment thereof may be selected from the group consisting of: Fv, Fab, F(ab′)₂, Fab′, scFv, scFv-Fc, minibody, nanobody and diabody.

In some embodiments the antibody or antigen-binding fragment thereof may comprise:
a heavy chain complementarity determining region 1 (CDR-H1) comprising the amino acid sequence: NEWMN (SEQ ID NO: 1) or KEWMN (SEQ ID NO: 2);
a heavy chain complementarity determining region 2 (CDR-H2) comprising the amino acid sequence: EIRLKSNNYATHYAESVKG (SEQ ID NO: 3);
a heavy chain complementarity determining region 3 (CDR-H3) comprising the amino acid sequence: DGWFAY (SEQ ID NO: 4);
a light chain complementarity determining region 1 (CDR-L1) comprising the amino acid sequence: RSSQSLLHSNGNTYVQ (SEQ ID NO: 5) or RSSQSLVHSNGNTYLN (SEQ ID NO: 6);
a light chain complementarity determining region 2 (CDR-L2) comprising the amino acid sequence: KVSNREP (SEQ ID NO: 7) or KVSNRFS (SEQ ID NO: 8); and
a light chain complementarity determining region 3 (CDR-L3) comprising the amino acid sequence: AQSTHVPRT (SEQ ID NO: 9) or SQSTHEPRT (SEQ ID NO: 10).

In particular embodiments, the six CDR sequences may be selected according to the “91R” antibody disclosed in FIG. 9 of WO2015/075269 (incorporated herein by reference). In this case, the CDR sequences are as follows: CDR-H1 is SEQ ID NO: 1, CDR-H2 is SEQ ID NO: 3, CDR-H3 is SEQ ID NO: 4, CDR-L1 is SEQ ID NO: 5, CDR-L2 is SEQ ID NO: 7 and CDR-L3 is SEQ ID NO: 9.

In particular embodiments, the six CDR sequences may be selected according to the “92R” antibody disclosed in FIG. 9 of WO2015/075269 (incorporated herein by reference). In this case, the CDR sequences are as follows: CDR-H1 is SEQ ID NO: 2, CDR-H2 is SEQ ID NO: 3, CDR-H3 is SEQ ID NO: 4, CDR-L1 is SEQ ID NO: 6, CDR-L2 is SEQ ID NO: 8 and CDR-L3 is SEQ ID NO: 10.

In some embodiments the antibody or antigen-binding fragment thereof comprises:

a heavy chain variable region comprising the
amino acid sequence:
(SEQ ID NO: 11)
EVKLEDSGGGLVQPGRSMKLSCVASGFTFSNFWMNWVRQSPEKGLEWVA
EIRLKSNNYATHYAESVKGRFTISRDDSKSSVYLQMNNLRTEDTGIYYC
TSDGWFAYWGQGTLVTVSA
or
(SEQ ID NO: 12)
EVKLEESGGGLVQPGGSMKLSCVASGFTFNKFWMNWVRQSPEKGLEWVA
EIRLKSNNYATHYAESVKGRFTISRDDSKSSVYLQMNNLRAEDTGIYYC
ASDGWFAYWGQGTLVTVSA
or
(SEQ ID NO: 13)
EVQLVESGGGLVKPGGSLRLSCAASGFTFSKFWMNWVRQAPGKGLEWVG
EIRLKSNNYATHYAESVKGRFTISRDDSKNTLYLQMNSLKTEDTAVYYC
TSDGWFAYWGQGTLVTVSS;
and
a light chain variable region comprising the
amino acid sequence:
(SEQ ID NO: 14)
DVVMTQTPLSLPVSLGDQTSISCRSSQSLLHSNGNTYVQWYLRKPGQSP
KLLIYKVSNRFPGVPDRFSGSGSGTDFTFKISRVEAEDLGVYFCAQSTH
VPRTFGGGTKLEIKR
or
(SEQ ID NO: 15)
DVVMTQTPLSLPVSLGDQASISCRSSQSLVHSNGNTYLNWCLORPGQSP
KSLIYKVSNRFSGVPDRFSGSGSGTDFTLKISRVEAEDLGVYFCSQSTH
FPRTFGGGTKLEIKR
or
(SEQ ID NO: 16)
DVQMTQSPSSLSASVGDRVTITCRSSQSLVHSNGNTYLNWYQQKPGKAP
KLLIYKVSNRFSGVPSRFSGSGSGTDFTLTISSLQPEDFATYYCSQSTH
FPRTFGGGTKVEIK.

In particular, the antibody or antigen-binding fragment thereof may comprise the VH and VL of SEQ ID NOS: 11 and 14, or of SEQ ID NOs: 12 and 15, or of SEQ ID NOs: 13 and 16.

In some embodiments the antibody or antigen-binding fragment thereof exhibits a binding affinity dissociation constant K_{D} of 500 nM, 250 nM, 100 nM, 10 nM, or lower for human CCR9. The K_{D} value may be determined by any appropriate method. In particular, the K_{D} value may be as determined by Surface Plasmon Resonance (SPR) (e.g. BIACORE). In certain cases, the binding affinity constant K_{D} may be as determined by, or inferred from, whole cell-based antibody binding determination.

As used herein, the term “antibody molecule” of the present invention encompasses an antibody, or antigen-binding fragment thereof, not only full length antibodies (e.g., IgG), but also antigen-binding fragments thereof, for example, Fab, Fab′, F(ab′)₂, Fv fragments, human antibodies, humanised antibodies, chimeric antibodies, antibodies of a non-human origin, recombinant antibodies, and polypeptides derived from immunoglobulins produced by means of genetic engineering techniques, for example, single chain Fv (scFv), diabodies, heavy chain or fragments thereof, light chain or fragment thereof, VH or dimers thereof, VL or dimers thereof, Fv fragments stabilized by means of disulfide bridges (dsFv), molecules with single chain variable region domains (Abs), minibodies, scFv-Fc, and fusion proteins comprising an antibody, or any other modified configuration of the immunoglobulin molecule that comprises an antigen recognition site of a desired specificity. The antibody of the invention may also be a bispecific antibody. An antibody fragment may refer to an antigen binding fragment. An antibody includes an antibody of any class, namely IgA, IgD, IgE, IgG (or sub-classes thereof), and IgM, and the antibody need not be of any particular class. In addition, the antibody of the invention may be also conjugated to a further compound, such as a therapeutic agent, a toxin and the like.

In particular embodiments, the anti-CCR9 antibody or antigen-binding fragment thereof may be any anti-CCR9 antibody disclosed in WO2015/075269 (the contents of which are expressly incorporated herein by reference).

### Subject

The subject as used herein is mammalian, preferably human. The subject may be a non-human mammal, such as a domestic or farm animal or a laboratory animal. The subject may be male or female. The subject may be a patient. A subject may have been diagnosed with a disease or condition requiring treatment, may be suspected of having such a disease or condition, or may be at risk from developing such a disease or condition. In particular, the subject may have or be suspected of having or be at risk of developing a cancer. The subject may have been treated, or may be undergoing treatment, with anti-cancer therapy (e.g. with an anti-cancer drug treatment, surgical treatment and/or radiotherapy). In particular cases, the cancer of the subject may have relapsed, spread and/or developed resistance to a first-line therapy. The subject may, in particular, have a blood neoplasia. The subject may, in particular, have T-cell acute lymphoblastic leukemia (T-ALL), T-cell lineage lymphomas, or acute myeloid leukemia (AML).

### Combination Therapy

As described in detail herein, the present inventors have found that combination treatment of tumor-bearing mice with a CCR9-targeted antibody (SRB1) and the chemotherapy agent vincristine resulted in significantly increased median survival time not only in comparison with controls, but also in comparison with treatment groups that received only the SRB1 antibody or only vincristine. The results described herein show a synergistic relationship between SRB1 and vincristine in terms of anti-tumor efficacy. Moreover, the significant increase in median survival time was also achieved with a triple combination treatment using the SRB1 antibody, vincristine and the corticosteroid dexamethasone. Without wishing to be bound by any particular theory, the present inventors believe that the synergistic relationship on anti-tumor efficacy seen with the SRB1 antibody and vincristine will also be exhibited by anti-CCR9 antibodies in combination with other vinca alkaloids or in combination with other chemotherapeutic agents as specified herein.

Combination therapy as contemplated herein differs from the antibody-drug conjugate approach disclosed in WO2015/075269A1 because the combination therapy of the present invention includes an anti-CCR9 antibody molecule administered simultaneously, sequentially or separately with a chemotherapeutic agent as specified herein. That is, the combination therapy involves administration of at least two separate agents that are not conjugated or covalently linked to one another. The agents may be administered together as a mixture in a single pharmaceutical composition or they may be administered separately as two different pharmaceutical compositions. This has the advantage that the anti-CCR9 antibody molecule (which may for example be a naked anti-CCR9 antibody or an anti-CCR9 antibody-drug conjugate) may be administered at different time points, in different amounts or ratios or with different frequency from that of the chemotherapeutic agent. In specific embodiments contemplated herein, the frequency of administration of the chemotherapeutic agent may be greater than for the anti-CCR9 antibody molecule.

In some embodiments, the dosing schedule in humans for the anti-CCR9 antibody molecule and the chemotherapeutic agent, e.g. vincristine, may be as follows:

Following clinical guidelines, the administration of anti-CCR9 antibody immunotherapy is usually performed intravenously, by mouth or by injection, in 4 cycles in fixed doses of between 200 and 400 mg/week. For vincristine, recommended dosage schedule is up to 2 mg/m² per week, in 4 cycles by intravenous infusion (Gilbar, Chambers et al. 2015; Hoelzer, Bassan et al. 2016; Majem, Juan et al. 2019; Mikhael, Ismaila et al. 2019; (CADTH) 2019; Compendium-EMC 2020; England-NHS 2020; (ASHP) updated 2020).

The chemotherapeutic agent or agents for combination therapy with the anti-CCR9 antibody molecule may be selected from the group consisting of: docetaxel, paclitaxel, nanoparticle albumin-bound paclitaxel, vinblastine or vincristine. In some embodiments the chemotherapeutic agent comprises a vinka alkaloid, such as vincristine, vinblastine, vindesine, vinorelbine, vincaminol, vineridine, and vinburnine.

In some embodiments the compositions for use and methods of the present invention further comprises a corticosteroid such as dexamethasone. In some embodiments both a vinka alkaloid (e.g. vincristine) and a corticosteroid (e.g. dexamethasone) are administered with the anti-CCR9 antibody molecule.

### Pharmaceutical Compositions and Treatment

The anti-CCR9 antibody molecule and pharmaceutically acceptable compositions thereof of the present invention may be administered to patients by any number of different routes, including intravenous, cutaneous or subcutaneous, nasal, intramuscular, transepithelial, intraperitoneal and oral administration.

The compositions of the invention may be formulated as pharmaceutical compositions that may be in the forms of solid or liquid compositions. Such compositions will generally comprise a carrier of some sort, for example a solid carrier or a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the anti-CCR9 antibody molecule of the present invention.

In addition to one or more of the anti-CCR9 antibody molecules of the present invention, optionally in combination with another active ingredient, the compositions can comprise one or more of a pharmaceutically acceptable excipient, carrier, buffer, stabiliser, isotonicising agent, preservative or anti-oxidant or other materials well known to those skilled in the art. Such materials should be non-toxic and should not interfere with the efficacy of the active ingredient. The precise nature of the carrier or other material may depend on the route of administration.

Preferably, the pharmaceutically compositions are given to an individual in a prophylactically effective amount or a therapeutically effective amount (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Handbook of Pharmaceutical Additives, 2nd Edition (eds. M. Ash and I. Ash), 2001 (Synapse Information Resources, Inc., Endicott, New York, USA); Remington's Pharmaceutical Sciences, 20th Edition, 2000, pub. Lippincott, Williams & Wilkins; and Handbook of Pharmaceutical Excipients, 2nd edition, 1994. By way of example, the compositions are preferably administered to patients in dosages of between about 0.01 and 100 mg of active compound per kg of body weight, and more preferably between about 0.5 and 10 mg/kg of body weight.

The following is presented by way of example and is not to be construed as a limitation to the scope of the claims.

### EXAMPLES

### Methodology

In Vivo Efficacy of Anti-CCR9 Antibody SRB1 in NSG Mice with MOLT4-GFP Xenografts—Combination of SRB1 with Chemotherapy Agents

NSG mice were injected into the vein of the tail with 1×10⁶ MOLT4-GFP cells in 100 μl of PBS by injection intravenous (day 0) and received 4 doses of monoclonal antibody (SRB1 or isotypic control at one-week intervals, days 4, 10, 17 and 24) of 400 μg each (16 mg/kg). Animals were sacrificed on day 28. The number of tumor total cells in spleen and bone marrow was determined by fluorescence microscopy, after an analysis by flow cytometry where the tumor cells were detected by GFP expression. The presence of accumulations of tumor cells in the spleen of these mice was also analyzed.

Furthermore, the effect of SRB1 treatment on survival of animals with MOLT4-GFP xenografts (10 animals/group) was determined by using SRB1 (4 mg/kg) or isotypic control, administered in 4 doses (once a week). Besides, other chemotherapeutic agents were used: Vincristine: 0.6 mg/kg/mouse and Dexamethasone: 50 μg/dose/mouse, following this protocol:
MOLT4-GFP cells were injected intravenously (1×10⁶ cells/mouse) on day 0.
Treatment with intravenous vincristine (0.6 mg/kg) on day 5.
Treatment with dexamethasone (50 μg/mouse/dose) days 5-9 (both included). This treatment was given alone or in combination with vincristine.
Treatment with SRB1 (100 μg/mouse/dose) days 11, 18, 25 and 32 intraperitoneally.
Treatment with isotypic control (100 μg/mouse/dose) days 11, 18, 25 and 32 intraperitoneally.

Results were analyzed by Kaplan-Meyer test (see further details herein).

### Example 1—In Vivo Survival of Orthotopic Mouse Models Following Treatment with Specified Antibody-Drug Combinations

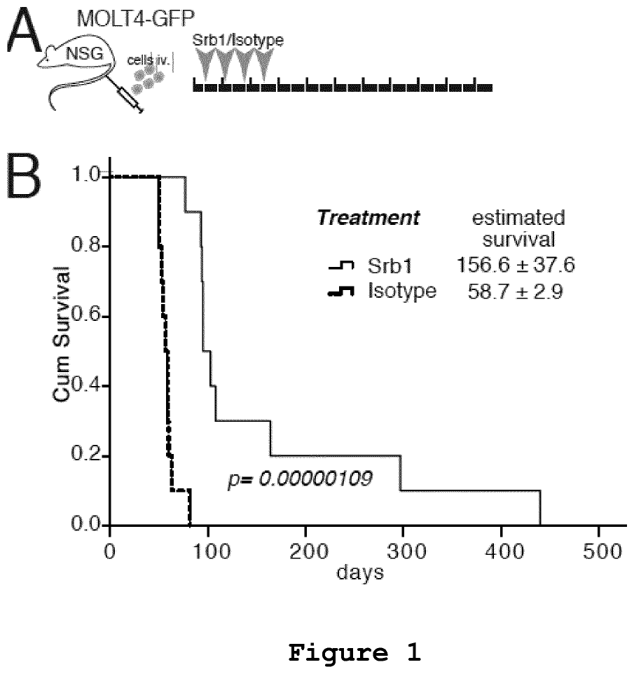

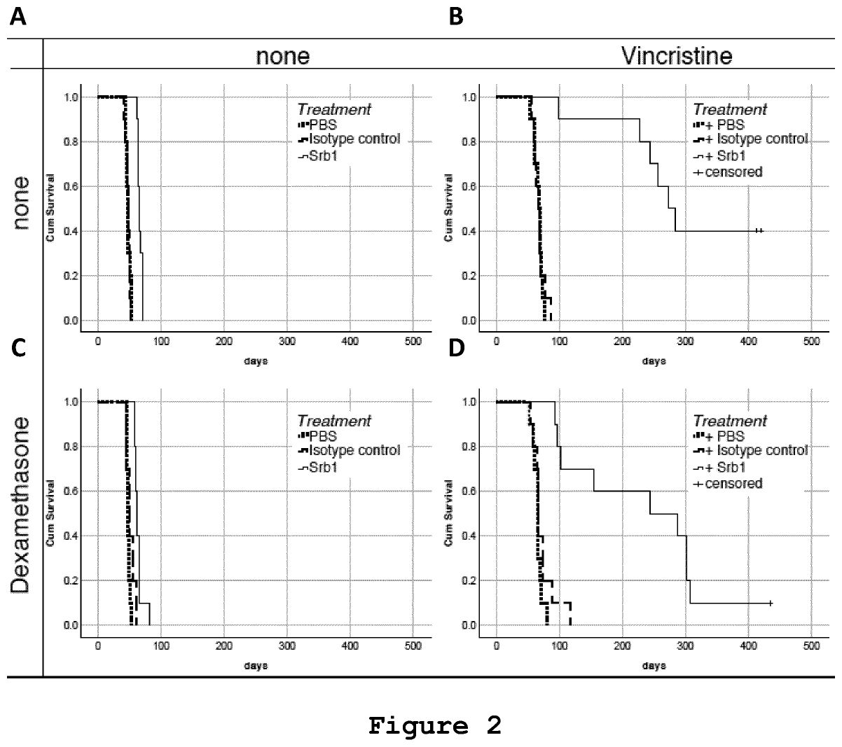

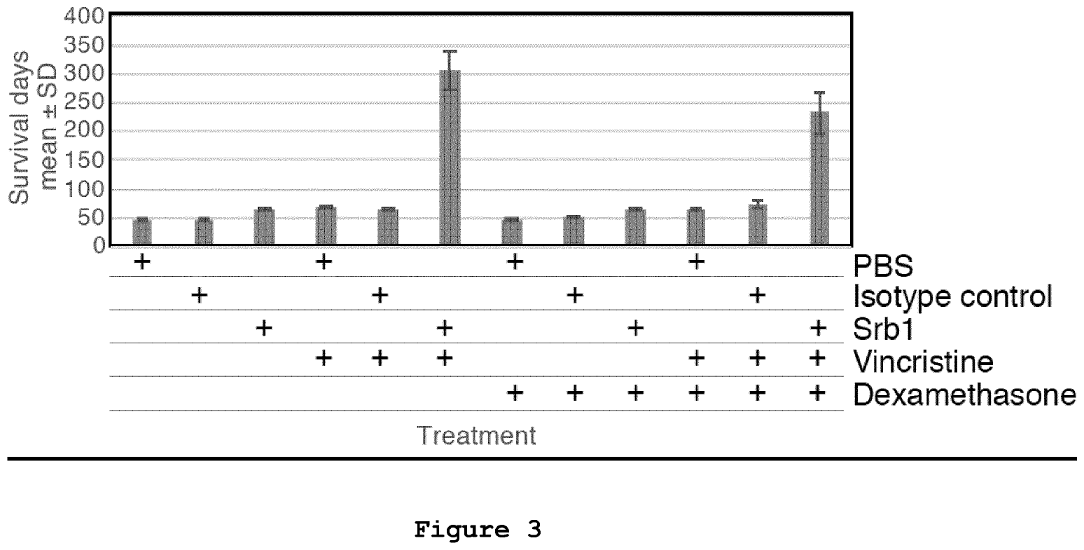

As shown in FIGS. 1-3 and the following table, an increased survival of animals was observed when using SRB1 at 4 mg/kg vs Control (IC50 70 days vs 48 days). Combination of SRB1 with the vinca alkaloid, vincristine, lengthens the survival of mice to a median survival time of 306.2 days (“Vincristine+ SRB1”). Moreover, the triple combination SRB1+vincristine+dexamethasone also exhibited elevated survival with a median survival time of 232.1 days.

TABLE

| | | | | | | | | | | VX + | | VX + |
|---|---|---|---|---|---|---|---|---|---|---|---|---|
| | SRB1 | | | VX + | | VX + | DX + | | DX + | DX + | VX + | DX + |
| | (4 mg/kg | Isotype | Vehicle | SRB1 | VX | Isotype | SRB1 | DX | Isotype | SRB1 | DX | Isotype |
| # censored | 10 | 10 | 10 | 4 | 10 | 10 | 10 | 10 | 10 | 10 | 10 | 10 |
| subjects | | | | | | | | | | | | |
| # deaths | 10 | 10 | 10 | 6 | 10 | 10 | 10 | 10 | 10 | 9 | 10 | 10 |
| Median | 66.2 | 47.1 | 47.6 | 306.2 | 67.9 | 66.6 | 63.3 | 47.7 | 51.1 | 232.1 | 65.5 | 72.9 |
| survival | | | | | | | | | | | | |
| (days) | | | | | | | | | | | | |

It is apparent that the combination of SRB1 and vincristine significantly lengthens median survival time beyond mere additive effects on survival time seen with SRB1 or vincristine administered alone (306.2 days vs. 66.2 days and 67.9 days, respectively). The present results therefore demonstrate a synergistic combination of anti-CCR9 antibody (SRB1) and vinca alkaloid therapeutic (vincristine) with or without additional corticosteroid therapy (dexamethasone) for the treatment of cancer, particularly acute lymphoblastic leukaemia (ALL).

All references cited herein are incorporated herein by reference in their entirety and for all purposes to the same extent as if each individual publication or patent or patent application was specifically and individually indicated to be incorporated by reference in its entirety.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

### REFERENCES

(ASHP), A. S. o. H.-S. P. (updated 2020). AHFS Monographs: Vincristine.
(CADTH), C. A. f. D. a. T. i. H. (2019). Dosing and Timing of Immuno-Oncology Drugs. Technology Review: Optimal Use 360 Report Version 1.1. Amersi, F. F., A. M. Terando, Y. Goto, R. A. Scolyer, J. F. Thompson, A. N. Tran, M. B. Faries, D. L. Morton and D. S. Hoon (2008). “Activation of CCR9/CCL25 in cutaneous melanoma mediates preferential metastasis to the small intestine.” Clin Cancer Res 14 (3): 638-645.
Bachelerie, F., A. Ben-Baruch, A. M. Burkhardt, C. Combadiere, J. M. Farber, G. J. Graham, R. Horuk, A. H. Sparre-Ulrich, M. Locati, A. D. Luster, A. Mantovani, K. Matsushima, P. M. Murphy, R. Nibbs, H. Nomiyama, C. A. Power, A. E. Proudfoot, M. M. Rosenkilde, A. Rot, S. Sozzani, M. Thelen, O. Yoshie and A. Zlotnik (2014). “International Union of Basic and Clinical Pharmacology. [corrected]. LXXXIX. Update on the extended family of chemokine receptors and introducing a new nomenclature for atypical chemokine receptors.” Pharmacol Rev 66 (1): 1-79.
Berx, G. and F. van Roy (2009). “Involvement of members of the cadherin superfamily in cancer.” Cold Spring Harb Perspect Biol 1 (6): a003129.
Borst, J., T. Ahrends, N. Babała, C. J. M. Melief and W. Kastenmüller (2018). “CD4 (+) T cell help in cancer immunology and immunotherapy.” Nat Rev Immunol 18 (10): 635-647.
Brightman, S. E., M. S. Naradikian, A. M. Miller and S. P. Schoenberger (2020). “Harnessing neoantigen specific CD4 T cells for cancer immunotherapy.” J Leukoc Biol 107 (4): 625-633.
Carramolino, L., A. Zaballos, L. Kremer, R. Villares, P. Martin, C. Ardavin, A. C. Martínez and G. Márquez (2001). “Expression of CCR9 beta-chemokine receptor is modulated in thymocyte differentiation and is selectively maintained in CD8 (+) T cells from secondary lymphoid organs.” Blood 97 (4): 850-857.
Cavallaro, U. and G. Christofori (2004). “Cell adhesion and signalling by cadherins and Ig-CAMs in cancer.” Nat Rev Cancer 4 (2): 118-132.
Chen, H., X. Cong, C. Wu, X. Wu, J. Wang, K. Mao, J. Li, G. Zhu, F. Liu, X. Meng, J. Song, X. Sun, X. Wang, S. Liu, S. Zhang, X. Yang, Y. Song, Y. G. Yang and T. Sun (2020). “Intratumoral delivery of CCL25 enhances immunotherapy against triple-negative breast cancer by recruiting CCR9 (+) T cells.” Sci Adv 6 (5): eaax4690.
Compendium-EMC, E. M. (2020). Vincristine: Summary of Product Characteristics.
DeVries, M. E., A. A. Kelvin, L. Xu, L. Ran, J. Robinson and D. J. Kelvin (2006). “Defining the origins and evolution of the chemokine/chemokine receptor system.” J Immunol 176 (1): 401-415.
England-NHS, N. H. S. i. (2020). Chemotherapy Protocol for Lung Cancer: Carboplatin (AUC6)-Paclitaxel-Pembrolizumab. Version 1.1.
Gilbar, P., C. R. Chambers and M. Larizza (2015). “Medication safety and the administration of intravenous vincristine: international survey of oncology pharmacists.” J Oncol Pharm Pract 21 (1): 10-18.
Gupta, P., P. K. Sharma, H. Mir, R. Singh, N. Singh, G. H. Kloecker, J. W. Lillard, Jr. and S. Singh (2014). “CCR9/CCL25 expression in non-small cell lung cancer correlates with aggressive disease and mediates key steps of metastasis.” Oncotarget 5 (20): 10170-10179.
Heinrich, E. L., A. K. Arrington, M. E. Ko, C. Luu, W. Lee, J. Lu and J. Kim (2013). “Paracrine Activation of Chemokine Receptor CCR9 Enhances The Invasiveness of Pancreatic Cancer Cells.” Cancer Microenviron 6 (3): 241-245.
Hoelzer, D., R. Bassan, H. Dombret, A. Fielding, J. M. Ribera and C. Buske (2016). “Acute lymphoblastic leukaemia in adult patients: ESMO Clinical Practice Guidelines for diagnosis, treatment and follow-up.” Ann Oncol 27 (suppl 5): v69-v82.
Hu, Y., L. Zhang, R. Wu, R. Han, Y. Jia, Z. Jiang, M. Cheng, J. Gan, X. Tao and Q. Zhang (2011). “Specific killing of CCR9 high-expressing acute T lymphocytic leukemia cells by CCL25 fused with PE38 toxin.” Leuk Res 35 (9): 1254-1260.
Johnson, E. L., R. Singh, C. M. Johnson-Holiday, W. E. Grizzle, E. E. Partridge, J. W. Lillard, Jr. and S. Singh (2010). “CCR9 interactions support ovarian cancer cell survival and resistance to cisplatin-induced apoptosis in a PI3K-dependent and FAK-independent fashion.” J Ovarian Res 3:15.
Johnson-Holiday, C., R. Singh, E. Johnson, S. Singh, C. R. Stockard, W. E. Grizzle and J. W. Lillard, Jr. (2011). “CCL25 mediates migration, invasion and matrix metalloproteinase expression by breast cancer cells in a CCR9-dependent fashion.” Int J Oncol 38 (5): 1279-1285.
Khandelwal, N., M. Breinig, T. Speck, T. Michels, C. Kreutzer, A. Sorrentino, A. K. Sharma, L. Umansky, H. Conrad, I. Poschke, R. Offringa, R. König, H. Bernhard, A. Machlenkin, M. Boutros and P. Beckhove (2015). “A high-throughput RNAi screen for detection of immune-checkpoint molecules that mediate tumor resistance to cytotoxic T lymphocytes.” EMBO Mol Med 7 (4): 450-463.
Kunkel, E. J., J. J. Campbell, G. Haraldsen, J. Pan, J. Boisvert, A. I. Roberts, E. C. Ebert, M. A. Vierra, S. B. Goodman, M. C. Genovese, A. J. Wardlaw, H. B. Greenberg, C. M. Parker, E. C. Butcher, D. P. Andrew and W. W. Agace (2000). “Lymphocyte CC chemokine receptor 9 and epithelial chemokine thymus-expressed (TECK) expression distinguish the small intestinal immune compartment: Epithelial expression of tissue-specific chemokines as an organizing principle in regional immunity.” J Exp Med 192 (5): 761-768.
Lazennec, G. and A. Richmond (2010). “Chemokines and chemokine receptors: new insights into cancer-related inflammation.” Trends Mol Med 16 (3): 133-144.
Letsch, A., U. Keilholz, D. Schadendorf, G. Assfalg, A. M. Asemissen, E. Thiel and C. Scheibenbogen (2004). “Functional CCR9 expression is associated with small intestinal metastasis.” J Invest Dermatol 122 (3): 685-690.
Majem, M., O. Juan, A. Insa, N. Reguart, J. M. Trigo, E. Carcereny, R. Garcia-Campelo, Y. Garcia, M. Guirado and M. Provencio (2019). “SEOM clinical guidelines for the treatment of non-small cell lung cancer (2018).” Clin Transl Oncol 21 (1): 3-17.
Mikhael, J., N. Ismaila, M. C. Cheung, C. Costello, M. V. Dhodapkar, S. Kumar, M. Lacy, B. Lipe, R. F. Little, A. Nikonova, J. Omel, N. Peswani, A. Prica, N. Raje, R. Seth, D. H. Vesole, I. Walker, A. Whitley, T. M. Wildes, S. W. Wong and T. Martin (2019). “Treatment of Multiple Myeloma: ASCO and CCO Joint Clinical Practice Guideline.” J Clin Oncol 37 (14): 1228-1263.
Mirandola, L., M. Chiriva-Internati, D. Montagna, F. Locatelli, M. Zecca, M. Ranzani, A. Basile, M. Locati, E. Cobos, W. M. Kast, R. Asselta, E. M. Paraboschi, P. Comi and R. Chiaramonte (2012). “Notch1 regulates chemotaxis and proliferation by controlling the CC-chemokine receptors 5 and 9 in T cell acute lymphoblastic leukaemia.” J Pathol 226 (5): 713-722.
Mittal, R., A. P. Patel, L. H. Debs, D. Nguyen, K. Patel, M. Grati, J. Mittal, D. Yan, P. Chapagain and X. Z. Liu (2016). “Intricate Functions of Matrix Metalloproteinases in Physiological and Pathological Conditions.” J Cell Physiol 231 (12): 2599-2621.
Mukaida, N. and T. Baba (2012). “Chemokines in tumor development and progression.” Exp Cell Res 318 (2): 95-102.
Qiuping, Z., L. Qun, H. Chunsong, Z. Xiaolian, H. Baojun, Y. Mingzhen, L. Chengming, H. Jinshen, G. Qingping, Z. Kejian, S. Zhimin, Z. Xuejun, L. Junyan and T. Jinquan (2003). “Selectively increased expression and functions of chemokine receptor CCR9 on CD4+ T cells from patients with T-cell lineage acute lymphocytic leukemia.” Cancer Res 63 (19): 6469-6477.
Raffetto, J. D. and R. A. Khalil (2008). “Matrix metalloproteinases and their inhibitors in vascular remodeling and vascular disease.” Biochem Pharmacol 75 (2): 346-359.
Raman, D., T. Sobolik-Delmaire and A. Richmond (2011). “Chemokines in health and disease.” Exp Cell Res 317 (5): 575-589.
Sarvaiya, P. J., D. Guo, I. Ulasov, P. Gabikian and M. S. Lesniak (2013). “Chemokines in tumor progression and metastasis.” Oncotarget 4 (12): 2171-2185.
Sharma, P. K., R. Singh, K. R. Novakovic, J. W. Eaton, W. E. Grizzle and S. Singh (2010). “CCR9 mediates PI3K/AKT-dependent antiapoptotic signals in prostate cancer cells and inhibition of CCR9-CCL25 interaction enhances the cytotoxic effects of etoposide.” Int J Cancer 127 (9): 2020-2030.
Singh, R., C. R. Stockard, W. E. Grizzle, J. W. Lillard, Jr. and S. Singh (2011). “Expression and histopathological correlation of CCR9 and CCL25 in ovarian cancer.” Int J Oncol 39 (2): 373-381.
Singh, S., U. P. Singh, J. K. Stiles, W. E. Grizzle and J. W. Lillard, Jr. (2004). “Expression and functional role of CCR9 in prostate cancer cell migration and invasion.” Clin Cancer Res 10 (24): 8743-8750.
Thorpe, L. M., H. Yuzugullu and J. J. Zhao (2015). “PI3K in cancer: divergent roles of isoforms, modes of activation and therapeutic targeting.” Nat Rev Cancer 15 (1): 7-24.
Tu, Z., R. Xiao, J. Xiong, K. M. Tembo, X. Deng, M. Xiong, P. Liu, M. Wang and Q. Zhang (2016). “CCR9 in cancer: oncogenic role and therapeutic targeting.” J Hematol Oncol 9:10.
Weitzenfeld, P. and A. Ben-Baruch (2014). “The chemokine system, and its CCR5 and CXCR4 receptors, as potential targets for personalized therapy in cancer.” Cancer Lett 352 (1): 36-53.
Wendland, M., N. Czeloth, N. Mach, B. Malissen, E. Kremmer, O. Pabst and R. Forster (2007). “CCR9 is a homing receptor for plasmacytoid dendritic cells to the small intestine.” Proc Natl Acad Sci USA 104 (15): 6347-6352.
Wu, W., N. Doan, J. Said, D. Karunasiri and S. T. Pullarkat (2014). “Strong expression of chemokine receptor CCR9 in diffuse large B-cell follicular correlates with lymphoma and lymphoma strongly gastrointestinal involvement.” Hum Pathol 45 (7): 1451-1458.
Wurbel, M. A., B. Malissen and J. J. Campbell (2006). “Complex regulation of CCR9 at multiple discrete stages of T cell development.” Eur J Immunol 36 (1): 73-81.
Yoon, S. O., S. J. Park, C. H. Yun and A. S. Chung (2003). “Roles of matrix metalloproteinases in tumor metastasis and angiogenesis.” J Biochem Mol Biol 36 (1): 128-137.
Youn, B. S., C. H. Kim, F. O. Smith and H. E. Broxmeyer (1999). “TECK, an efficacious chemoattractant for human thymocytes, uses GPR-9-6/CCR9 as a specific receptor.” Blood 94 (7): 2533-2536.
Zaballos, A., J. Gutiérrez, R. Varona, C. Ardavin and G. Márquez (1999). “Cutting edge: identification of the orphan chemokine receptor GPR-9-6 as CCR9, the receptor for the chemokine TECK.” J Immunol 162 (10): 5671-5675.
Zabel, B. A., W. W. Agace, J. J. Campbell, H. M. Heath, D. Parent, A. I. Roberts, E. C. Ebert, N. Kassam, S. Qin, M. Zovko, G. J. LaRosa, L. L. Yang, D. Soler, E. C. Butcher, P. D. Ponath, C. M. Parker and D. P. Andrew (1999). “Human G protein-coupled receptor GPR-9-6/CC chemokine receptor 9 is selectively expressed on intestinal homing T lymphocytes, mucosal lymphocytes, and thymocytes and is required for thymus-expressed chemokine-mediated chemotaxis.” J Exp Med 190 (9): 1241-1256.
Zhang, Z., T. Sun, Y. Chen, S. Gong, X. Sun, F. Zou and R. Peng (2016). “CCL25/CCR9 Signal Promotes Migration and Invasion in Hepatocellular and Breast Cancer Cell Lines.” DNA Cell Biol 35 (7): 348-357.
Zlotnik, A. and O. Yoshie (2000). “Chemokines: a new classification system and their role in immunity.” Immunity 12 (2): 121-127.

1-16. (canceled)

17. A method of treatment of cancer in a mammalian subject, comprising administering simultaneously, sequentially or separately to the subject in need thereof a therapeutically effective amount of an anti-CCR9 antibody molecule and a chemotherapeutic agent selected from the group consisting of: vincristine, docetaxel, paclitaxel, nanoparticle albumin-bound paclitaxel, and vinblastine,
wherein said anti-CCR9 antibody molecule and said chemotherapeutic agent are not conjugated together.

18. The method of claim 17, wherein said anti-CCR9 antibody molecule comprises a monoclonal antibody or antigen-binding fragment thereof that specifically binds to CCR9.

19. The method of claim 17, wherein said chemotherapeutic agent comprises vincristine.

20. The method of claim 17, wherein the cancer is a blood neoplasia.

21-22. (canceled)

23. The method of claim 17, further comprising simultaneously, sequentially or separately administering dexamethasone to the subject.

24. The method of claim 18, wherein the anti-CCR9 monoclonal antibody or antigen-binding fragment thereof is selected from the group consisting of: Fv, Fab, F(ab′)₂, Fab′, scFv, scFv-Fc, minibody, nanobody and diabody.

25. The method of claim 18, wherein the monoclonal antibody or antigen-binding fragment thereof comprises:
a heavy chain complementarity determining region 1 (CDR-H1) comprising the amino acid sequence: NFWMN (SEQ ID NO: 1) or KFWMN (SEQ ID NO: 2);
a heavy chain complementarity determining region 2 (CDR-H2) comprising the amino acid sequence: EIRLKSNNYATHYAESVKG (SEQ ID NO: 3);
a heavy chain complementarity determining region 3 (CDR-H3) comprising the amino acid sequence: DGWFAY (SEQ ID NO: 4);
a light chain complementarity determining region 1 (CDR-L1) comprising the amino acid sequence: RSSQSLLHSNGNTYVQ (SEQ ID NO: 5) or RSSQSLVHSNGNTYLN (SEQ ID NO: 6);
a light chain complementarity determining region 2 (CDR-L2) comprising the amino acid sequence: KVSNRFP (SEQ ID NO: 7) or KVSNRFS (SEQ ID NO: 8); and
a light chain complementarity determining region 3 (CDR-L3) comprising the amino acid sequence: AQSTHVPRT (SEQ ID NO: 9) or SQSTHFPRT (SEQ ID NO: 10).

26. The method of claim 25, wherein the monoclonal antibody or antigen-binding fragment thereof comprises:
a heavy chain variable region comprising the
amino acid sequence:
(SEQ ID NO: 11)
EVKLEDSGGGLVQPGRSMKLSCVASGFTFSNFWMNWVRQSPEKGLEWVA
EIRLKSNNYATHYAESVKGRFTISRDDSKSSVYLQMNNLRTEDTGIYYC
TSDGWFAYWGQGTLVTVSA
or
(SEQ ID NO: 12)
EVKLEESGGGLVQPGGSMKLSCVASGFTFNKFWMNWVRQSPEKGLEWVA
EIRLKSNNYATHYAESVKGRFTISRDDSKSSVYLQMNNLRAEDTGIYYC
ASDGWFAYWGQGTLVTVSA
or
(SEQ ID NO: 13)
EVQLVESGGGLVKPGGSLRLSCAASGFTFSKFWMNWVRQAPGKGLEWVG
EIRLKSNNYATHYAESVKGRFTISRDDSKNTLYLQMNSLKTEDTAVYYC
TSDGWFAYWGQGTLVTVSS;
and
a light chain variable region comprising the
amino acid sequence:
(SEQ ID NO: 14)
DVVMTQTPLSLPVSLGDQTSISCRSSQSLLHSNGNTYVQWYLRKPGQSP
KLLIYKVSNRFPGVPDRFSGSGSGTDFTFKISRVEAEDLGVYFCAQSTH
VPRTFGGGTKLEIKR
or
(SEQ ID NO: 15)
DVVMTQTPLSLPVSLGDQASISCRSSQSLVHSNGNTYLNWCLQRPGQSP
KSLIYKVSNRFSGVPDRFSGSGSGTDFTLKISRVEAEDLGVYFCSQSTH
FPRTFGGGTKLEIKR
or
(SEQ ID NO: 16)
DVQMTQSPSSLSASVGDRVTITCRSSQSLVHSNGNTYLNWYQQKPGKAP
KLLIYKVSNRFSGVPSRFSGSGSGTDFTLTISSLQPEDFATYYCSQSTH
FPRTFGGGTKVEIK.

27. The method of claim 17, wherein the antibody molecule exhibits a binding affinity dissociation constant K_{D} of 500 nM, 250 nM, 100 nM, 10 nM, or lower for human CCR9, as determined by Surface Plasmon Resonance (SPR).

28. The method of claim 17, wherein the antibody molecule exhibits CCR9 specific binding in the presence of 10 μg/mL concentration of human CCL25, as measured by flow cytometry.

29. The method of claim 17, wherein the antibody molecule exhibits CCR9+ lymphocyte depletion.

30. The method of claim 17, wherein said chemotherapeutic agent comprises vincristine and wherein said method further comprises simultaneously, sequentially or separately administering dexamethasone.

31. The method of claim 17, wherein the cancer is T-cell acute lymphoblastic leukemia (T-ALL), T-cell lineage lymphomas, or acute myeloid leukemia (AML).


SEQUENCE LISTING (16 sequences)

SEQ ID NO: 1 - protein, 5 aa, synthetic construct
NFWMN

SEQ ID NO: 2 - protein, 5 aa, synthetic construct
KFWMN

SEQ ID NO: 3 - protein, 19 aa, synthetic construct
EIRLKSNNYATHYAESVKG

SEQ ID NO: 4 - protein, 6 aa, synthetic construct
DGWFAY

SEQ ID NO: 5 - protein, 16 aa, synthetic construct
RSSQSLLHSNGNTYVQ

SEQ ID NO: 6 - protein, 16 aa, synthetic construct
RSSQSLVHSNGNTYLN

SEQ ID NO: 7 - protein, 7 aa, synthetic construct
KVSNRFP

SEQ ID NO: 8 - protein, 7 aa, synthetic construct
KVSNRFS

SEQ ID NO: 9 - protein, 9 aa, synthetic construct
AQSTHVPRT

SEQ ID NO: 10 - protein, 9 aa, synthetic construct
SQSTHFPRT

SEQ ID NO: 11 - protein, 117 aa, synthetic construct
EVKLEDSGGGLVQPGRSMKLSCVASGFTFSNFWMNWVRQSPEKGLEWVAEIRLKSNNYATHYAESVKGRFTISRDDSKSSVYLQMNNLRTEDTGIYYCTSDGWFAYWGQGTLVTVSA

SEQ ID NO: 12 - protein, 117 aa, synthetic construct
EVKLEESGGGLVQPGGSMKLSCVASGFTFNKFWMNWVRQSPEKGLEWVAEIRLKSNNYATHYAESVKGRFTISRDDSKSSVYLQMNNLRAEDTGIYYCASDGWFAYWGQGTLVTVSA

SEQ ID NO: 13 - protein, 117 aa, synthetic construct
EVQLVESGGGLVKPGGSLRLSCAASGFTFSKFWMNWVRQAPGKGLEWVGEIRLKSNNYATHYAESVKGRFTISRDDSKNTLYLQMNSLKTEDTAVYYCTSDGWFAYWGQGTLVTVSS

SEQ ID NO: 14 - protein, 113 aa, synthetic construct
DVVMTQTPLSLPVSLGDQTSISCRSSQSLLHSNGNTYVQWYLRKPGQSPKLLIYKVSNRFPGVPDRFSGSGSGTDFTFKISRVEAEDLGVYFCAQSTHVPRTFGGGTKLEIKR

SEQ ID NO: 15 - protein, 113 aa, synthetic construct
DVVMTQTPLSLPVSLGDQASISCRSSQSLVHSNGNTYLNWCLQRPGQSPKSLIYKVSNRFSGVPDRFSGSGSGTDFTLKISRVEAEDLGVYFCSQSTHFPRTFGGGTKLEIKR

SEQ ID NO: 16 - protein, 112 aa, synthetic construct
DVQMTQSPSSLSASVGDRVTITCRSSQSLVHSNGNTYLNWYQQKPGKAPKLLIYKVSNRFSGVPSRFSGSGSGTDFTLTISSLQPEDFATYYCSQSTHFPRTFGGGTKVEIK